SEQUENCE LISTING (7 sequences)

SEQ ID NO: 1 - DNA, 309 bp, Thunnus thynnus thynnus
  misc_feature: BFT-FSH-12C1N
gggcagggttgcagttacggctgtcatccaaagaacatcagcatctctgtggagagctgtggcatcaccgagttcatcctcaccaccatatgtgaaggacagtgctacctcgaggatccggtctacatcagccatgatgagcagaagatctgtaatggcgactggtcctatgaggtgaaacacatcgaaggctgtccggtgggagtcacctaccctgtggccagaaactgcgagtgtaccacgtgtaacacaggaaacacgtactgcgggcgccttcccggatacgtacccagctgtccgtccttttaa

SEQ ID NO: 2 - DNA, 309 bp, Thunnus thynnus thynnus
  misc_feature: BFT-FSH-12C0N
gggcagggttgcagttacggctgtcatccaaagaacatcaggatctctgtggagagctgtggcatcaccgagttcatcctcaccaccatatgtgaaggacagtgctacctcgaggatccggtctacatcagccatgatgagcagaagatctgtaatggcgactggtcctatgaggtgaaacacatcgaaggctgtccggtgggagtcacctaccctgtggccagaaactgcgagtgtaccacgtgtaacacaggaaacacgtactgcgggcgccttcccggatacgtacccagctgtccgtccttttaa

SEQ ID NO: 3 - DNA, 309 bp, Thunnus thynnus thynnus
  misc_feature: BFT-FSH-12C2N
ggtcaaggatgttcctacggttgtcacccaaagaacatctccatttccgttgagtcctgtggtatcactgagttcatcaacactaccatatgtgagggtcagtgttacttggaggacccagtttacatttctcacgacgagcagaagatttgtaacggagactggtcctacgaagttaagcacatcgagggttgtccagttggtgttacttacccagttgctagaaactgtgagtgtactacttgtaacactggtaacacttactgtggtagattgccaggttacgtaccatcttgtccatctttttag

SEQ ID NO: 4 - DNA, 321 bp, Thunnus thynnus thynnus
  misc_feature: BFT-FSH-13C1N
ggtcaaggatgttcctacggttgtcacccaaagaacatctccatttccgttgaatctgaggagtgtggttcctgtatcactgagttcatcttgactaccatatgtgagggtcagtgttacttggaggacccagtttacatttctcacgacgagcagaagatttgtaacggagactggtcctacgaagttaagcacatcgagggttgtccagttggtgttacttacccagttgctagaaactgtgagtgtactacttgtaacactggtaacacttactgtggtagattgccaggttacgtaccatcttgtccatctttttag

SEQ ID NO: 5 - DNA, 321 bp, Thunnus thynnus thynnus
  misc_feature: BFT-FSH-13C0N
ggtcaaggatgttcctacggttgtcacccaaagaacatcagaatctccgttgaatctgaggaatgtggttcctgtatcactgagttcatcttgactaccatatgtgagggtcagtgttacttggaggacccagtttacatttctcacgacgagcagaagatttgtaacggagactggtcctacgaagttaagcacatcgagggttgtccagttggtgttacttacccagttgctagaaactgtgagtgtactacttgtaacactggtaacacttactgtggtagattgccaggttacgtaccatcttgtccatctttttag

SEQ ID NO: 6 - DNA, 315 bp, Thunnus thynnus thynnus
  misc_feature: BFT-FSH-13C2N
ggtcaaggatgttcctacggttgtcacccaaagaacatctccatttccgttgaatctgaggagtgtggttcctgtatcactttcaacactaccatatgtgagggtcagtgttacttggaggacccagtttacatttctcacgacgagcagaagatttgtaacggagactggtcctacgaagttaagcacatcgagggttgtccagttggtgttacttacccagttgctagaaactgtgagtgtactacttgtaacactggtaacacttactgtggtagattgccaggttacgtaccatcttgtccatctttttag

SEQ ID NO: 7 - DNA, 285 bp, Thunnus thynnus thynnus
  misc_feature: GP-alpha
tacccaaacactgacttgtccaacatgggttgtgaggcttgtactttgagaaagaacactgttttctccagagacagaccaatctaccagtgtatgggatgttgtttctccagagcttacccaactccattgaaggctatgaaaactatgactatcccaaagaacatcacttccgaggctacttgttgtgttgctaagcacgtttacgagactgaagttgctggtatcagagttagaaaccacactgactgtcactgttccacttgttactaccacaagatctag

(19) United States
(12) Patent Application Publication
(10) Pub. No.: US 2007/0281883 A1
(43) Pub. Date: Dec. 6, 2007

(54) DEVELOPMENT OF FOLLICLE STIMULATING HORMONE AGONISTS AND ANTAGONISTS IN FISH

(76) Inventors: Hanna Rosenfeld, Eilat (IL); Shalom Zemach, Kfar Yona (IL)

Correspondence Address: DR. MARK M. FRIEDMAN;C/O BILL POLKINGHORN - DISCOVERY DISPATCH, 9003 FLORIN WAY, UPPER MARLBORO, MD, 20772, US

(21) Appl. No.: 11/422,091

(22) Filed: Jun. 5, 2006

(51) Int. Cl. C07K 14/59; C07H 21/04

(52) U.S. Cl. 514/8; 530/397; 435/69.4; 435/320.1; 435/325; 536/23.5

(57) ABSTRACT

The invention provides recombinant forms of piscine follicle-stimulating hormone (FSH) with characteristic intramolecular disulfide bonds and modified glycosylation patterns in the β-subunit that enhance the stability and metabolic activity of the hormone. Also provided are recombinant materials to produce the FSH β and glycoprotein α-subunits singly or in combination to obtain complete heterodimeric hormone of regulated glycosylation pattern. The piscine FSH agonists of the invention are therapeutically useful to expedite the onset of puberty in captive fish and to alleviate reproductive dysfunctions in fish. Likewise, the piscine FSH antagonists of the invention will be therapeutically useful to halt gonadal development, thereby contributing to body weight gain of the fish,

### FIELD OF THE INVENTION

The present invention relates to the production of modified forms of piscine glycoprotein hormone, namely, follicle-stimulating, hormone (FSH), for use in marine and freshwater aquaculture. In particular, it concerns production of recombinant forms of piscine FSH with modified glycosylation patterns and activities, with characteristic intramolecular disulfide bonds and glycosylation patterns in the FSHβ subunit that enhance the stability and metabolic activity of the hormones The specific modifications are obtained by site directed mutagenesis at the appropriate amino acid residues. The modified forms of piscine FSH are agonist to the corresponding native piscine FSH and can combine with receptors for piscine FSH to produce a physiologic reaction typical of the naturally occurring piscine FSH. The piscine FSH agonists of the invention are therapeutically useful to expedite the onset of puberty and to alleviate reproductive dysfunctions in captive fish The modified forms of piscine FSH may also exhibit antagonist activity and be useful to retard sexual development in fish, which will contribute to the overall growth performance of the fish.

### BACKGROUND

FSH is a key regulator of gonadal function and is widely applied in assisted reproductive technologies. In most vertebrates, the hormone has a dominant role in the initiation of gametogenesis and regulation of gonadal growth (i.e. spermatogenesis in males and follicle growth and maturation in females),

FSH is a glycoprotein composed of two subunits, α and β. Within a given animal species, the α-subunit is common to other glycoprotein hormones (hereinafter termed glycoprotein α subunit [GPα]), including chorionic gonadotropin (CG), luteinizing hormone (LH), and thyroid-stimulating hormone (TSH), whereas the β-subunit is hormone specific. Both subunits exhibit high content of cysteine residues (10 in GPα and 12 in the β subunits) forming multiple intramolecular disulfide bonds known to determine the tertiary structure of the molecule. In addition, each subunit contains N-linked glycosylation sequons (Asn-X-Ser/Thr), two in the GPα- and FSHβ subunits, and one in the LHβ subunit. The N-linked oligosaccharide contains an N-acetylglucosamine residue at its reducing terminal and is linked to an amide group of an Asn residue of a polypeptide In addition to the two consensus sites for N-linked glycosylation, CGβ exhibits four O-linked glycosylation structures (N-acetylgalactosamine residue, which is linked to the hydroxyl group of either a serine or threonine residue of a polypeptide) in its cauboxyl terminal extension Carbohydrates are therefore highly important for structural as well as functional characteristics of the protein molecules (i.e. folding, subunit assembly, heterodimer secretion, interaction with the specific receptor, and metabolic clearance rate).

FSHβ has been sequenced in representative species of all vertebrates including seven fish orders (Yaron et al., (2003) International Review of Cytology—a Survey of Cell Biology, 225: 131-185). Since the primary structure of human FSH was determined, human FSH is used therapeutically to regulate facets of human female reproduction. Analogs to human FSH have been made for use in sterilization, conception and other therapeutic and clinical applications in humans. Genomic clones and isolates for human FSHβ have been prepared (Watkins, P. C. et al. DNA (1987) 6:205-212; Jameson, J. L. et al., Mol Endocrinol (1988) 2:806-815; Jameson, J. L. et al. J Clin Endocrinol Metab (1986) 64:319-327; Glaser, T. et al. Nature (1986) 321:882-887), and humam FSHβ has been engineered to permit recombinant production of the hormone. Boime et al. in U.S. Pat. Nos. 5,338,835; 5,705,478; 6,737,515; 6,689,365; 6,693,074; and 6,306,654, among others, disclose mutein forms of human FSH with potential therapeutic human reproductive applications.

While there is some similarity between reproduction among vertebrates, given the evolutionary distance between fish and mammals, reproduction in mammals and fish is significantly different in terms of structure and function. Mating approaches including behavior; genetics, gamete structure, sperm-egg recognition and interaction, mechanism of fertilization, developmental biology and embryology, among other aspects, differ greatly, both physiologically and biochemically, between fish and mammalian species.

Aquaculture of marine and freshwater food fish or shellfish has succeeded in developing high yields in terms of rearing fish. However, most fish in captivity do not experience the conditions of their natural spawning grounds and, consequently, fail to reproduce spontaneously (Zohar and Mylonas, Aquaculture (2001) 197:99-136). Fish may encounter fertility difficulties at various stages of the reproductive process Either the fish do not progress through gonadal development, or female broodstock do not progress through final oocyte maturation and development. Alternatively, there may be an absence of spawning at the end of the reproductive cycle.

Over the years, hormonal approaches employing gonadotropin-releasing hormone (GnRH), LH enriched preparations (fleshly ground pituitaries of reproductively mature fish), and LH-like agents (human CG), have been used to overcome problems halting final stages of ovarian maturation and spawning (Mylonas and Zohar, Rev Fish Biol Fisher (2001) 10:463-491). Nevertheless, the available therapeutic agents fall short of dealing with induction of early stages of gonadal growth (i.e. vitellogenesis and spermatogenesis). As a result, many commercially important fish (freshwater eels, Mediterranean amberjack, etc.) exhibiting a complete failure to undergo vitellogenesis/spermatogenesis are not available for aquaculture uses, and their production is based merely on fishery of wild stocks. In this regard, once available, administration of FSH, the major regulator and initiator of gonadal growth, can solve such reproductive dysfunctions.

FSH preparations can also be used to accelerate the onset of puberty, a process by which an immature fish acquires the capacity to reproduce for the first time. The control of puberty in piscine is of great importance for fish farming, as it takes many years to achieve maturity in various species. For example, fish such as the black caviar producing paddlefish, groupers and tunas grow to a relatively large size over several years (>5) before their onset of puberty. It takes time, feed, labor, and space costs to produce and maintain stocks for these species. Therefore, the ability to accelerate the onset of puberty, induction of precocious maturation, and obtainment of fertilized eggs at a desired time in these species, would greatly improve the cost-efficiency of fish farming operations.

### Unique Structural Trials of Teleost FSHβ

Among piscine, teleosts (bony fishes) constitute the largest and most diverse division of vertebrates, with over 22,000 known species. Comparison of vertebrate FSHβ sequences demonstrates a high degree of similarity among tetrapods, and a considerable variability among piscine, and particularly teleost fish. FIG. 1A shows a multiple sequence alignment comparing the amino acid sequences of previously identified tetrapods and piscine FSHβ and illustrates the regions of identity and conserved structural motifs.

Sequences are aligned from the first deduced amino acid of the signal peptide. Gaps (denoted by dashes) were introduced to maximize alignment. The conserved 12 cysteine residues are marked with white letters on black background and are numbered counting from the N-terminal. The additional cysteine within teleost sequences is numbered as “C₋₁”. The sequons encoding putative N-linked glycosylation sites are marked with gray background and numbered as N₁ and N₂. Identification of species and gene bank accession Nos./references for the selected FSHβ sequences shown in FIG. 1 are presented in Table 1 below.

TABLE 1
Identification of Tetrapod and Piscine Sequences Shown in FIG. 1

| | | | Gene Bank |
|---|---|---|---|
| | | | Accession No./ |
| | Species | Abbreviation | Reference |
| tetrapods | Cow | Bov | M13383 |
| | Human | Hum | M5491-3 |
| | Horse | Hor | AB029157 |
| | Mouse | Mo | U12932 |
| | Quail | Qu | U41406 |
| | Chicken | Chic | BI392995 |
| | Turtle | Tur | AB085201 |
| | Frog | Fr | AB178054 |
| piscine | Dogfish | Df | AJ310344 |
| | Sturgeon | Stu | AJ251658 |
| | European eel | Ee | AY169722 |
| | Japanese eel | Je | AB016169 |
| | Conger eel | Ce | AJ271632 |
| | Japanese conger | Cj | AB045157 |
| | Canal catfish | Ccf | AF112191 |
| | Goldfish | Gf | D88023 |
| | Common carp | Cca | AB003583 |
| | Black carp | Bca | AF319961 |
| | Masu salmon | Ms | S69275 |
| | Rainbow trout | Rt | AB050835 |
| | Gilthead seabream | Sb | Elizur et al. (1996) Gen. |
| | | | Comp. Endocrinol. |
| | | | 102: 39–46 |
| | Striped bass | Stb | L35070 |
| | Tilapia | T | AF289174 |
| | Bluefin tuna | BFT | Unpublished |
| | | | (see FIG. 4B herein) |
| | Killifish | Kf | M87014 |
| | Atlantic halibut | AtH | AJ417768 |

FIG. 1B is a composite evolutionary tree of several of the selected species of Table 1 showing FSHβ divergence. The tree was constructed by the maximum-parsimony method based on amino acid sequences of the preprotein (signal mid mature protein). The values at the nodes are bootstrap probabilities (%) estimated by 100 replications. Teleost lineages are shown highlighted in grey background, i.e., the orders Ostariophysi, Perciformes/Pleuronectiformes, Salmoniformes, and Siluroniformes. Arrows indicate branches leading to the 12 and 13 cysteine backbone typifying teleost FSHβ.

TABLE 2
Percentage Similarity Between FSHβ Amino Acid Sequences

| | Ms | Rt | Bca | Cca | Gf | Cj | Co | Je | Ee | Kf | T | AtH | Sb | Stb |
|---|---|---|---|---|---|---|---|---|---|---|---|---|---|---|
| Hum | 36.3 | 36.3 | 40.8 | 42.4 | 43.2 | 40.8 | 38.3 | 42.9 | 44.3 | 30.6 | 27.3 | 32.0 | 31.6 | 31.7 |
| Hor | 36.3 | 36.3 | 43.2 | 44.8 | 45.6 | 41.7 | 39.2 | 44.5 | 46.0 | 32.4 | 30.0 | 34.5 | 34.2 | 34.2 |
| Bov | 37.1 | 37.1 | 44.8 | 46.4 | 47.2 | 43.3 | 40.8 | 44.4 | 45.9 | 32.4 | 29.1 | 34.5 | 33.3 | 33.3 |
| Mo | 38.4 | 38.4 | 40.5 | 41.3 | 42.1 | 42.2 | 40.5 | 45.7 | 47.2 | 32.2 | 27.9 | 32.5 | 31.4 | 32.2 |
| Tur | 34.1 | 33.3 | 36.0 | 33.4 | 39.2 | 37.5 | 36.7 | 40.7 | 42.6 | 31.6 | 31.9 | 29.1 | 32.5 | 31.6 |
| Fr | 32.2 | 33.1 | 33.1 | 33.9 | 34.7 | 35.4 | 34.5 | 37.5 | 38.1 | 30.9 | 32.1 | 30.1 | 30.1 | 31.9 |
| Df | 35.8 | 37.6 | 37.9 | 39.7 | 40.6 | 40.6 | 39.6 | 43.8 | 45.4 | 34.0 | 35.1 | 35.2 | 35.6 | 36.5 |
| Stu | 38.2 | 38.3 | 39.7 | 42.1 | 41.3 | 42.2 | 40.5 | 45.6 | 46.4 | 33.9 | 33.3 | 29.8 | 32.2 | 33.9 |
| Bft | 44.4 | 44.4 | 42.8 | 45.3 | 43.6 | 40.4 | 37.7 | 43.0 | 43.0 | 64.0 | 70.0 | 65.8 | 71.8 | 63.8 |
| Stb | 42.4 | 42.4 | 44.2 | 46.7 | 45.0 | 41.0 | 38.5 | 41.0 | 41.0 | 62.3 | 62.0 | 67.5 | 75.9 | — |
| Sb | 41.5 | 41.6 | 40.9 | 45.0 | 44.2 | 40.2 | 38.5 | 38.5 | 38.5 | 57.0 | 57.5 | 65.8 | — | |
| AtH | 39.9 | 39.9 | 43.0 | 46.3 | 46.3 | 39.8 | 38.2 | 39.1 | 41.5 | 53.6 | 56.7 | — | | |
| T | 44.3 | 43.4 | 43.4 | 45.1 | 43.4 | 38.2 | 36.4 | 36.4 | 36.4 | 54.6 | — | | | |
| Kf | 41.1 | 41.1 | 39.5 | 41.2 | 39.5 | 42.9 | 42.0 | 39.3 | 39.3 | — | | | | |
| Ee | 38.7 | 38.0 | 52.0 | 54.3 | 55.1 | 72.0 | 73.6 | 99.9 | — | | | | | |
| Je | 37.5 | 36.7 | 52.0 | 54.3 | 55.2 | 72.0 | 73.6 | — | | | | | | |
| Ce | 34.4 | 33.6 | 44.0 | 46.4 | 47.2 | 95.2 | — | | | | | | | |
| Cj | 35.3 | 34.4 | 44.8 | 46.4 | 47.2 | — | | | | | | | | |
| Gf | 42.5 | 41.7 | 85.4 | 95.4 | — | | | | | | | | | |
| Cca | 44.1 | 43.3 | 86.9 | — | | | | | | | | | | |
| Bca | 43.3 | 42.5 | — | | | | | | | | | | | |
| Rt | 96.4 | — | | | | | | | | | | | | |
| Ms | — | | | | | | | | | | | | | |

| | Bft | Stu | Df | Fr | Tur | Mo | Bov | Hor | Hum |
|---|---|---|---|---|---|---|---|---|---|
| Hum | 30.8 | 49.6 | 47.4 | 62.0 | 71.3 | 86.0 | 88.4 | 91.5 | — |
| Hor | 33.3 | 51.2 | 50.0 | 54.5 | 69.8 | 84.5 | 91.5 | — | |
| Bov | 32.5 | 49.7 | 45.6 | 52.9 | 69.0 | 86.1 | — | | |
| Mo | 32.2 | 51.2 | 46.5 | 54.5 | 69.0 | — | | | |
| Tur | 32.5 | 46.5 | 50.0 | 59.4 | — | | | | |
| Fr | 31.9 | 43.1 | 58.5 | — | | | | | |
| Df | 35.7 | 47.0 | — | | | | | | |
| Stu | 34.8 | — | | | | | | | |
| Bft | — | | | | | | | | |
| Stb | | | | | | | | | |
| Sb | | | | | | | | | |
| AtH | | | | | | | | | |
| T | | | | | | | | | |
| Kf | | | | | | | | | |
| Ee | | | | | | | | | |
| Je | | | | | | | | | |
| Ce | | | | | | | | | |
| Cj | | | | | | | | | |
| Gf | | | | | | | | | |
| Cca | | | | | | | | | |
| Bca | | | | | | | | | |
| Rt | | | | | | | | | |
| Ms | | | | | | | | | |

Table 2 shows the percentage similarity between the FSHβ amino acid sequences of FIG. 1 computed from a pairwise distance matrix analysis. Accordingly, the homology rate between tetrapod and perciform FSHβ sequences is lower than 35% (the respective values in Table 2 are marked with gray background) Recently, using window analysis of the nonsynonymous (dN) to synonymous (dS) mutation ratios along the protein sequence, we have provided evidence consistent with a directional selection acting to re-modulate teleost FSHβ molecule. FIG. 2 presents an example for such analyses, in which the d_{N}/d_{S} ratio was based on FSHβ sequences derived from the Japanese eel (Anguilla Japonica) and the striped bass (Morone saxatilis), representing the more extreme orders among teleosts, Anguilliformes and Perciformes, respectively. The ratio was significantly higher than 1 among the residues of the N-terminus, suggesting a positive selection underlying this particular subportion of the molecule,

Two general patterns typify the cysteine (“C”) residues backbone of teleost FSHβ: (i) incorporation of additional C (i.e. C₋₁ show in FIG. 1), which makes a total of 13 C residues. Such a pattern characterizes representatives of the superorder Ostariophysi (e.g., Black carp, canal catfish, common carp and goldfish), (ii) exclusion of C₃, and return to a scaffold based on 12 C residues (FIG. 1B). Such a pattern characterizes representatives of salmonid and perciform fish (e.g. Atlantic halibut, killifish, gilthead seabream, masu salmon, rainbow trout, striped bass, and tilapia), It is argued that the C residues alteration affects the formation of the “seatbelt” motif, and consequently, narrows the cap in the β-subunit through which the loop of the GPα-subunit is straddled (FIG. 3) This change is known to increase the stability of the teleost FSH heterodimer, which is the bioactive form (Xing et al., J Biol Chem (2004) 279:35449-35457). In terms of N-linked putative glycosylation sites, teleost FSHβ sequences exhibit all possible variations, i.e. two sites (as in mammals), single site (common to most teleosts), or die absence of such a site (seen in Atlantic halibut).

To date, therapeutic preparations of FSH (derived from human menopausal urine or recombinantly produced) are available only for the treatment of human infertility. Despite the knowledge of FSH in humans, there remains a widely recognized need in fish farming and marine aquaculture for therapeutic preparations to effectively manipulate reproduction in captive fish. It would be highly advantageous to develop FSH agonists and antagonists directed to therapeutic management of fish infertility, a major bottleneck in the development of commercial aquaculture, This will greatly contribute to efficient control of fish reproduction in marine and fresh water aquaculture.

### Unique Physiological Traits of Teleost FSHβ

In the mammalian ovary, FSH binds exclusively to FSH receptors (FSHRs) in granulosa cells, whereas LH binds its cognate receptors in thecal cells (Griswold et al., J. Steiroid Biochem Mol Biol (1995) 53:215-218) Nevertheless, this model describing “two cell types for two gonadotropins” was found to be inapplicable to teleost ovary. Ligand bindinig studies carried out with coho salmon vitellogenic oocytes defined similar binding sites to both FSH and LH within thecal and granulose layers (Miwa et al., Biology of Reproduction(1994) 50:629-642).

In mammals, the interactions of LH and FSH with cognate receptors are highly specific (Braun et al., Embo J (1991) 10:1885-1890; Tilly et al., Endocrinology, (1992) 130:1296-1302) However, the equivalent receptor-ligand interactions in teleosts demonstrate merely a loose specificity. All studies carried out so far with fish species including salmonids (Yan et al., Biol Reprod (1992) 47:418-427; Miwa et al., Biology of Reproduction (1994) 50:629-642; Oba et al., Fish Physiology and Biochemistry (1999) 22:355-363; Oba et al., Biochemcial and Biophysical Research Communications (1999) 265:366-371; catfish (Bogerd et al., Biol Reprod (2001), 64:1633-1643; Vischer and Bogerd, Biology, of Reproduction (2003) 68:262-271; Vischer et al., Journal of Molecular Endocrinology (2003) 31:133-140; and zebrafish (So et al., Biol Reprod (2005) 72:1382-1396), show a similar trend, in which the FSH is capable of binding both FSH and LH albeit with some preference to the homologous ligand.

There is, therefore, considerable interest in the function of each residue in the FSH so that analogs can be designed with maximum efficiency as agonists mid antagonists to the FSH receptor, for use in pharmaceutical compositions in captive fish.

### OBJECTS AND SUMMARY OF THE INVENTION

It is therefore an object of the invention to provide recombinant forms of piscine FSH with characteristic intramolecular disulfide bonds and glycosylation patterns in the β-subunit that enhance the stability and metabolic activity of the hormone.

Another object of the invention is to produce bioactive recombinant agonists to piscine FSH useful in reproductive enhancement in captive fish.

A further object of the invention is to provide recombinant forms of piscine FSH that compete with the native hormones for binding to the ligand binding site of the receptor.

Yet another object of the invention is to provide teleost FSH analogs for therapeutic use in commercial fish farming to stimulate gonadal growth and regulate sexual maturation aid reproduction of captive fish.

Still another object of the invention is to provide piscine FSH analogs for use in manipulating the reproductive cycles and induce spawning of reared fish.

The invention provides recombinant forms of piscine follicle-stimulating hormone (FSH) that afford the opportunity to control the glycosylation pattern of beta portions of the heterodimer Such glycosylation control call be obtained by altering glycosylation sites by, for instance, site directed mutagenesis at the appropriate amino acid residues, or, alternatively, through selection of the recombinant eucaryotic host.

The invention provides recombinantly produced piscine FSH hormone with characteristic intramolecular disulfide bonds and preferred glycosylation patterns in the β-subunit of the heterodimer that enhances the stability and molecular activity of the hormones. The specific modifications are preferably obtained by site directed mutagenesis at the appropriate amino acid residues.

Thus, in one aspect, the invention is directed to specific mutants of piscine FSH hormone with characteristic intramolecular disulfide bonds and altered glycosylation patterns in the β-subunit, The FSH β-subunit may be prepared in nonglycosylated and partially glycosylated versions by disrupting the glycosylation sites normally present in the β-subunit. Similarly, surplus glycosylated versions of the hormones can be prepared by the addition of at least one putative N-linked or O-linked glycosylation site, which is not present normally in the FSHβ-subunit. Glycosylated versions are, of course, also included within the scope of the invention.

Thus, in this aspect, the invention is directed to three forms of FSHβ comprising the 12 cysteine backbone typifying teleosts with varying numbers of N-linked glycosylation sites (e.g., 12C0N, 12C1N, 12C2N), and three forms of FSHβ comprising the 13 cysteine backbone typifying cyprinids with varying numbers of N-linked glycosylation sites (e.g., 13C0N, 13C1N, 13C2N). Cells may be transfected simultaneously with two independent expression plasmids, one encompassing a selected form of FSHβ and the other encompassing the GPα-subunit (FIG. 6A), Alternatively, cells may be transfected with a single expression plasmid encompassing a translational-fusion of one of the aforementioned FSHβ forms and the GPα-subunit (FIG. 6B), i.e., a single-chain chimera.

In another, aspect, the invention is directed to expression systems capable, when transformed into a suitable host, of expressing the gene encoding muteins of the FSHβ subunit which have characteristic intramolecular disulfide bonds and modified glycosylation patterns, and to recombinant host cells transfected with these expression systems. In additional aspects, the invention is directed to recombinant hosts that have been transformed or transfected with this expression system, either singly, or in combination with an expression system capable of producing the GPα-subunit. In other aspects, the invention is directed to piscine FSH beta monomers and piscine FSH heterodimers of defined glycosylation pattern produced by the recombinant host cells.

The FSH analogs produced can be used as agonists and may be useful as antagonists. The invention is directed also to the mutant piscine FSH glycoprotein with altered glycosylation or activity patterns produced by these cells.

In other aspects, the invention is directed to therapeutic or pharmaceutical compositions containing the recombinant forms of piscine FSH as set forth above for treating fertility in captive fish, and to methods to regulate reproductive metabolism in fish by administration of the recombinant forms of piscine FSH of the invention or pharmaceutical compositions containing them. The pharmaceutical composition includes as an active ingredient a physiologically effective amount of the mutant piscine FSH and a physiologically acceptable carrier, diluent, excipient and/or adjuvant.

In yet another aspect, the invention is directed to specific mutants of piscine FSH with altered glycosylation patterns in the beta subunit, or to beta subunit mutants containing alterations at the cysteine backbone, which effect intermolecular disulfide bond formation and enhance heterodimer stability. Thus, in another aspect, the invention is directed to expression systems for the piscine FSH beta subunit and its mutants which lack glycosylation sites at the asparagine at position N₁ or position N₂ or both (FIG. 1), mutants which have additional N-linked or O-linked glycosylation sites, and to recombinant host cells transfected with these expression systems. The cells may be transfected with a subunit expression system singly or in combination with an expression system for piscine glycoprotein alpha subunit.

The invention provides recombinant forms of piscine FSH with characteristic disulfide bonds and defined glycosylation pattern in the beta-subunit. The piscine FSHβ may either be surplus glycosylated, fully glycosylated, partially glycosylated, or nonglycosylated. The resulting FSH agonists retain the activity of the unmodified heterodimeric form or are antagonists of this activity.

According to a further aspect of the present invention there is provided recombinantly produced piscine FSH by improved means that afford the ability to control the glycosylation pattern of the beta subunit.

Particularly preferred mutants are those where the glycosylation sites of the FSH β-subunit have been altered. Glycosylation patterns may be altered by destroying or reconstituting N-linked glycosylation sequons, and/or by the addition of at least one putative N-linked or O-linked glycosylation site, and/or by choice of host cell in which the protein is produced.

As described herein, one method of constructing effective piscine FSH agonists is to prepare recombinant piscine FSH β-subunit having 12 cysteine or 13 cysteine residues, to modify the natural N-linked glycosylation sites to two, single, or no glycosylation sites (FIG. 5), and to insert within the FSH β-subunit sequence additional N-linked or O-linked glycosylation site/s, and thus affect the agonist or antagonist activity of the piscine FSH glycoprotein. Mutants of the FSH β-subunit in which the N-linked glycosylation site N_{1,} shown in FIG. 1) is eliminated by amino acid substitutions are preferred for agonist activity. Similar modifications at the glycosylation site at position N₂ (shown in FIG. 1) are also preferred. Particular mutants that are glycosylated or totally or partially de-glycosylated are set forth in FIG. 5.

Thus, in one aspect of the invention, there is provided a mutein of piscine follicle stimulating hormone (FSH) β-subunit having an at least 12 cysteine residue backbone and having a modified N-linked glycosylation pattern due to alteration of at least one N-linked glycosylation site of the native piscine FSH β-subunit nucleotide sequence, wherein the alteration is selected from the group consisting of a deletion of at least one N-linked glycosylation site and an addition of at least one N-linked glycosylation site. In another aspect of the invention, the mutein has a 13 cysteine residue backbone. In yet another embodiment of the invention, surplus glycosylated versions of the hormones can be prepared by the addition of at least one putative N-linked or O-linked glycosylation site, which is not present normally in the FSHβ-subunit.

In yet another aspect of the invention, there is provided a heterodimer comprising the mutein described above in combination with piscine glycoprotein α-subunit, wherein the heterodimer is an agonist or antagonist to the corresponding native piscine FSH gonadotropin hormone.

In another aspect, there is provided a method comprising utilizing piscine FSH to enhance fertility in captive fish, the improvement comprising substituting for the FSH the heterodimer comprising the mutein described above.

According to yet another aspect of the present invention there is provided a diagnostic kit for analysis of a biological sample removed from a piscine subject. The kit includes reagents suitable for conducting a quantitative analysis of a piscine FSH expression level in the biological sample.

Thus, in one aspect, the invention is directed to glycosylated, partially glycosylated, or nonglycosylated proteins that comprise the amino acid sequence of the piscine FSH β-subunit, singly or in combination with the piscine glycoprotein alpha subunit. In other aspects, the invention is directed to recombinant materials and methods to produce the proteins of the invention, to pharmaceutical compositions containing them, to antibodies specific to them, and to methods for their use.

### BRIEF DESCRIPTION OF THE DRAWINGS

The invention is herein described, by way of example only, with reference to the accompanying drawings and the following detailed description, it being understood that the particulars shown are by way of example and illustrative discussion only, and are presented to provide what is believed to be the most useful and readily understood description of the embodiments of the invention. No attempt is made to show structural details of the invention in more detail than is necessary for a fundamental understanding of the invention, the description taken with the drawings making apparent to those skilled in the art how the several forms of the invention may be embodied in practice.

In the Drawings:

FIG. 1A is a multiple sequence alignment comparing the amino acid sequences of previously identified tetrapods and piscine FSHβ and illustrating the regions of identity and conserved structural motifs including cysteine residues (C₋₁ to C₁₂) and putative N-linked glycosylation sites (N₁ and N₂).

FIG. 1B is a composite evolutionary tree of studied species showing FSHβ divergence. Teleost lineages are highlighted with grey background. Arrows indicate branches leading to the 12 and 13 cysteine backbone typifying teleost FSHβ. The tree was constructed by the maximum-parsimony method based on amino acid sequences of the preprotein (signal and mature protein). The values at the nodes are bootstrap probabilities (%) estimated by 100 replications.

FIG, 2 shows a sliding window analysis of the d_{N}/d_{S} ratio per site along the sequence codifying for FSHβ. Pairwise differences between two teleostean species, striped bass and Japanese eel, are shown. A dashed vertical line marks the threshold value (d_{N}/d_{S}=1) above which positive selection is inferred.

FIG. 3A shows multiple sequence alignment and structural motifs typifying teleost FSHβ (e.g. varying number of N-linked glycosylation sequons as well as varying number and position of cysteine residues). FSHβ sequences with 13 cysteine residues are capable of forming a “seatbelt” configuration either between C₁₂ and C₋₁ (full line) like most teleosts, or alternatively, between C₁₂ and C₃ (dashed line) like all tetrapods.

FIG. 3B is a schematic diagram of proposed tertiary structures of teleost (left panel) and tetrapod (right panel) FSHβ showing tentative disulfide bond pairings. Disulphide bonds forming the alternate “seatbelt” motifs are marked with gray lines, whereas all other disulphide bonds are marked with dashed lines. The “cystine knot” motif (circled) is formed by three disulfide bonds, which delineate an elongated structure of three β-hairpin loops (e.g. βL1, βL2, βL3). The N— and C-termini as well as subunit main loops are identified.

FIG. 4A shows cDNA sequences and deduced amino acid sequences of GPα-subunit. The N-terminal of the mature peptide was designated position +1 and the amino acids in the signal peptide are given negative numbers. The polyadenylation signal is underlined. The −1 and +1 signify the respective starting points and directions for signal and mature peptides.

FIG. 4B shows cDNA sequence and deduced amino acid sequence of bluefin tuna (BFT) FSHβ. The N-terminal of the mature peptide was designated position +1 and the amino acids in the signal peptide are given negative numbers. The polyadenylation signal is underlined. The −1 and +1 signify the respective starting points and directions for signal and mature peptides.

FIG. 4C shows the cDNA sequence encoding for a translational fusion consisting of mature BFT FSHβ and GPα peptides. The sequences encoding for FSHβ and GPα are highlighted with grey and black backgrounds, respectively. Additional sequences coding for 6-His Tag and restriction sites (EcoRI and NotI) are denoted.

FIG. 5A is a schematic illustration of native BFT FSHβ and mutant forms, i.e. native FSHβ—includes 12 cysteine residues and one N-linked glycosylation site (12C1N); mutant 1—includes 12 cysteine residues and totally lacks N-glycosylation sites (12C0N); mutant 2—includes 12 cysteine residues and two N-linked glycosylation sites (12C2N); mutant 3—includes 13 cysteine residues and lacks N-glycosylation sites (13C0N); mutant 4—includes 13 cysteine residues and one N-linked glycosylation site (13C1N); and mutant 5—includes 13 cysteine residues and two N-linked glycosylation sites (13C2N)

FIG. 5B represents amino acid sequences of native BFT-FSHβ and five mutant forms. Amino acids replaced and/or inserted by site direct mutations are shown as white letters on black background. Deleted amino acids are framed. The 6-His Tag insertion is highlighted with gray background. Sequons encoding for putative N-linked glycosylation sites are underlined. Mutant 1—includes a single replacement of serine (S) with arginine (R) disrupting the N-linked glycosylation sequon, NIS (N₁, shown in FIG. 1); Mutant 2—includes a single replacement of leucine (L) with asparagine (N) constituting an additional N-linked glycosylation sequon, NTT (N₂, shown in FIG. 1); Mutant 3—includes a replacement of (S) with (R) (as in mutant 1) and two insertions of di-amino acids: tandem glutamic acid [EE] and serine and cysteine [SC]. Both insertions mimic the corresponding motif in catfish and carp FSHβ which possess 13 cysteine residues; Mutant 4—includes two insertions of di-amino acids EE and SC (as in mutant 3); mutant 5—includes a replacement of L with N (as in mutant 2), two insertions of di-amino acids EE and SC (as in mutant 3 and mutant 4), and deletion of two amino acids: glutamic acid [E] and isoleucine [I].

FIG. 6A represents two independent plasmids for co-expression of BFT FSHβ- and GPα-subunits in Pichia pastoris. Each amplicon was inserted into the pPIC9K vector as an EcoRI/NotI insertion, downstream of the yeast mating factor-α secretion signal (S) sequence.

FIG. 6B represents a single plasmid encompassing a translational-fusion of BFT FSHβ and the GPα-subunit (FSHβα, a single-chain chimera). The α-F and α-R stand for primers listed in Table 3.

FIG. 7A shows commassie blue R-250 staining of recombinant BFT FSH heterodimers (consisting of the GPα-subunit and either BFT FSHβ 12C1N or 12C2N) and recombinant BFT FSHβ monomer (12C1N) that were separated on SDS-PAGE (10-20% gradient). Molecular mass marlkers (M) run simultaneously and their values in kDa appear on the left.

FIG, 7B shows immunodetection of recombinant BFT FSH forms (12C1N; 12C0N; 12C2N; 13C1N) and BFT pituitary FSH proteins. The proteins were separated on SDS-PAGE (10-20% gradienit) and analyzed by Western blotting using highly specific antibodies that were raised in rabbits against synthetic peptide coding for amino acids 50 to 65 of BFT-FSHβ. Molecular mass markers (M) run simultaneously and their values in kDa are indicated.

FIG. 8A is a bar graph showing the stimulatory effect of 50 ng/ml recombinant BFT FSH 12C1N analogs consisting of independent or fused FSHβ- and GPα-subunits ([α+β] or [βα], respectively), on estradiol secretion from ovarian follicles derived from vitellogenic mullet (Mugil cephalus). Controls were treated with fish saline Results are expressed as the means±SE (n=8). Means designated by the same letter are not significantly different (P>0.05, Tukey-Kramer Multiple Comparison Test).

FIG. 8B is a bar graph showing a dose-dependant stimulatory effect of recombinant BFT FSH analogs (e.g. 12C1N and 12C0N) on estradiol secretion from ovarian follicles derived from vitellogenic sea bass (Dicentrarchus labrax). Controls were treated with fish saline. Results are expressed as the means±SE (n=8). Means marked by different letters differ significantly (P<0.05, Tukey-Kramer Multiple Comparison Test). Both FSH analogs were found to be most effective at the lowest dose that was tested (0.5 ng/ml).

FIG. 9A shows BFT pituitary proteins separation on 2D-PAGE. Spots (A and B) enlightened by the anti-FSHβ are enclosed in circles. The corresponding positions of the molecular mass (kDa) markers (M) run simultaneously are indicated.

FIG. 9B shows 2D-PAGE Western blot analysis of BFT pituitary proteins. The spots (A and B) enlightened by the anti-FSHβ were robotically cut out from an equivalent 2D-PAGE stained with Commassie blue, and were subjected to Mass Spec analysis.

FIG. 9C shows BFT FSHβ deduced amino acid sequence. In frame is the synthetic peptide sequence (egg. BFT FSHβ amino acid residues 50 to 65) that was used as an antigen for the production of BFT-FSH specific antibodies in rabbits. Sequences of the most frequent peptides found among spots A and B, as analyzed by the Mass Spec technique, are shown in shadow.

### DETAILED DESCRIPTION OF THE PREFERRED EMBODIMENTS

The development of recombinant FSH agonists in fish according to the present invention may be better understood with reference to the accompanying figures, examples, and descriptions. It is contemplated that the invention is not limited in its application to the details set forth in the following description or drawings, or exemplified by the Examples. The invention may be practiced in various other ways and is capable of other embodiments. Also, it is contemplated that the phraseology and terminology used herein are for purposes of description and should not be regarded as limiting.

Generally, the terms and the laboratory procedures utilized in the present invention include molecular; biochemical, microbiological and recombinant DNA techniques which are thoroughly explained in the literature. See, for example, “Molecular Cloning: A Laboratory Manual” Sambrook et al., (1989); “Current Protocols in Molecular Biology” Volumes I-III Ausubel, R. M., ed, (J 994); Ausubel et al, “Current Protocols in Molecular Biology”, John Wiley and Sons, Baltimore. Md. (1989); Perbal, “A Practical Guide to Molecular Cloning”, John Wiley & Sons, New York (1988); Watson et al, “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4.666,828; 4,683,202; 4,801,531; 5,192,659 and 5,272,057; “Cell Biology: A Laboratory Handbook” Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait. M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

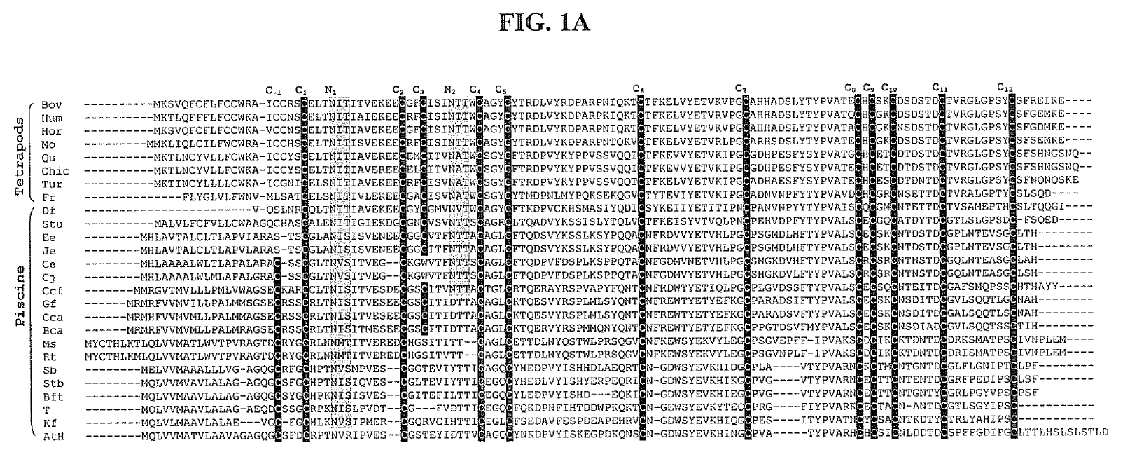

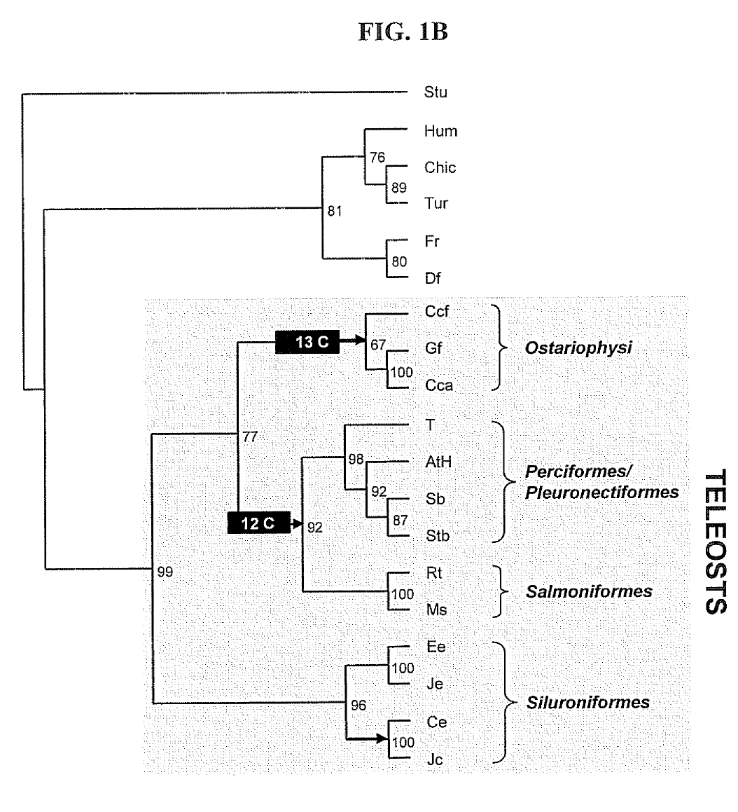

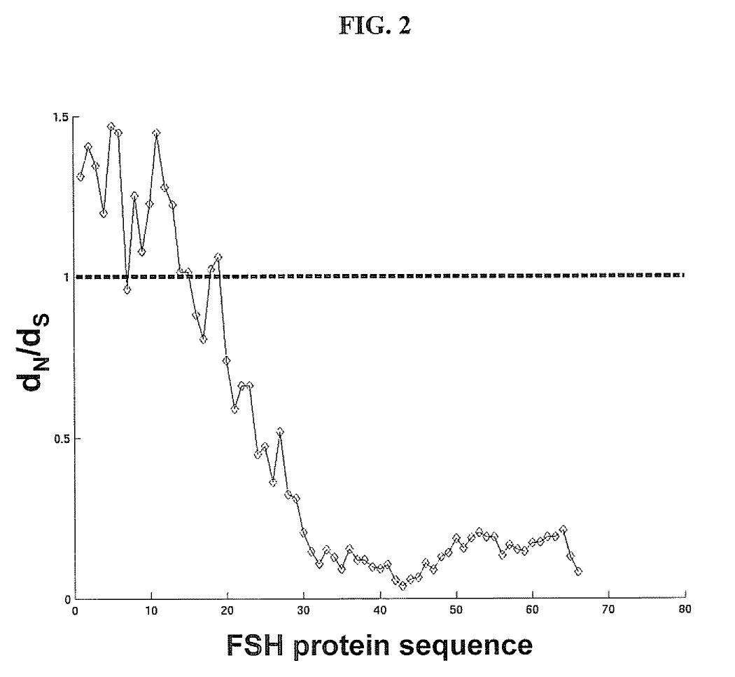

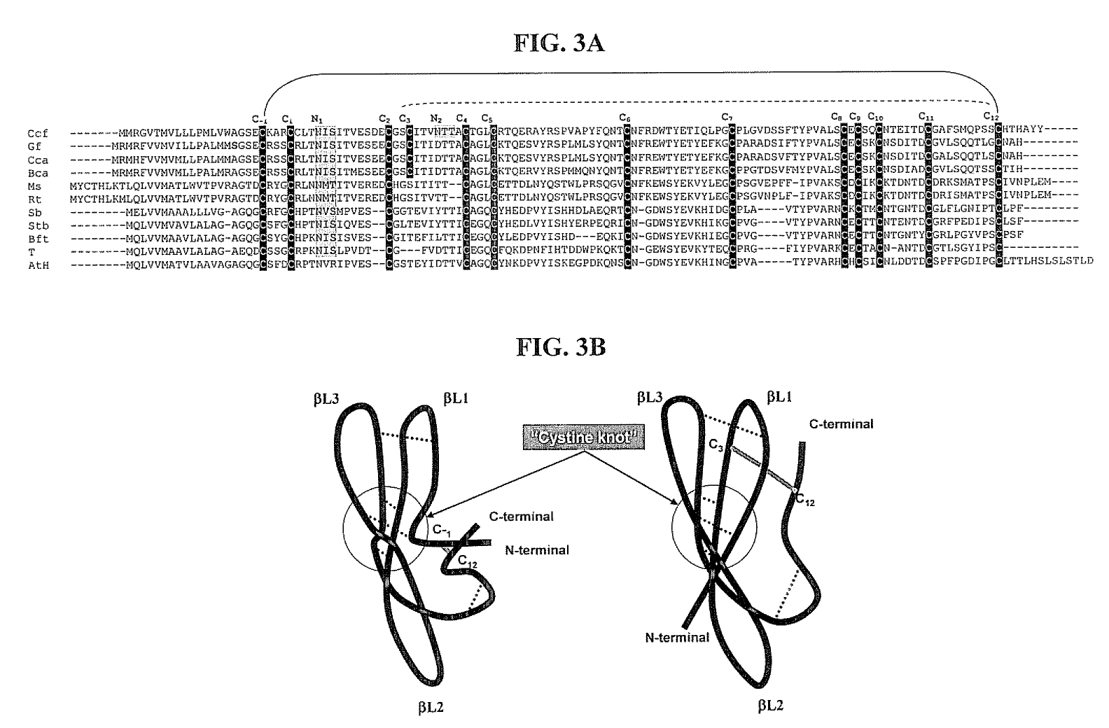

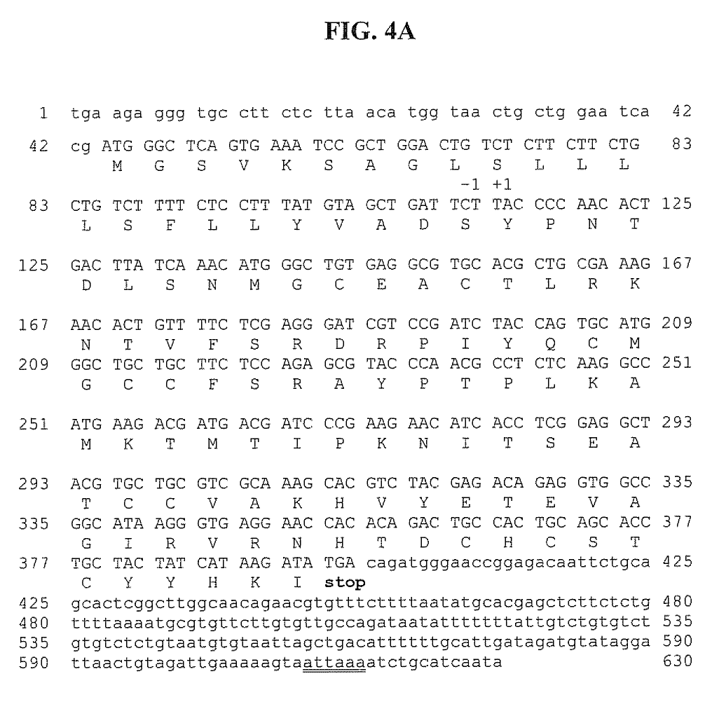

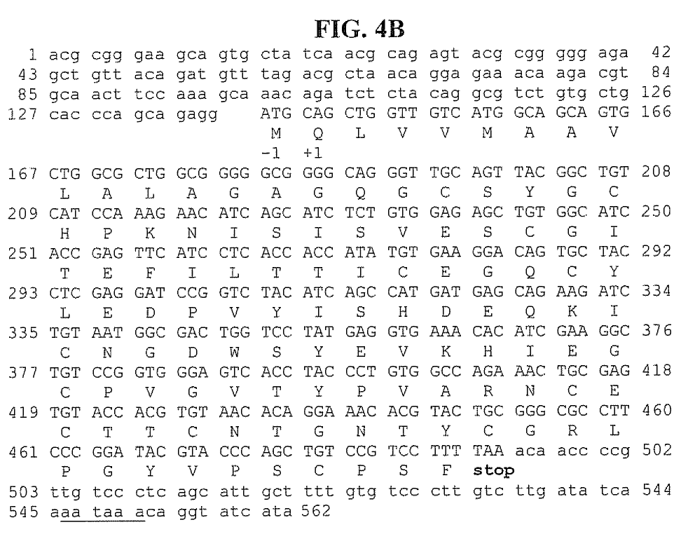

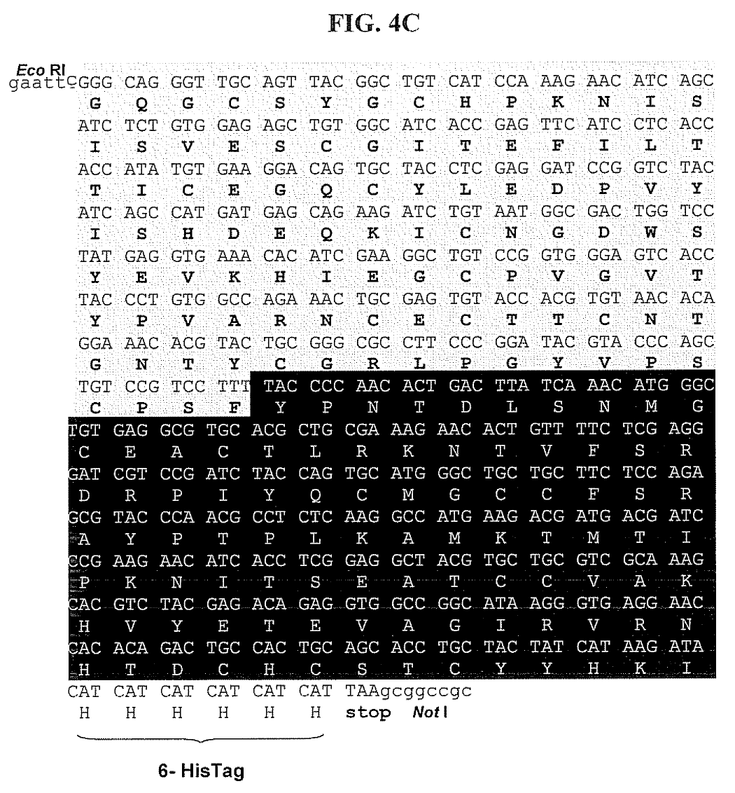

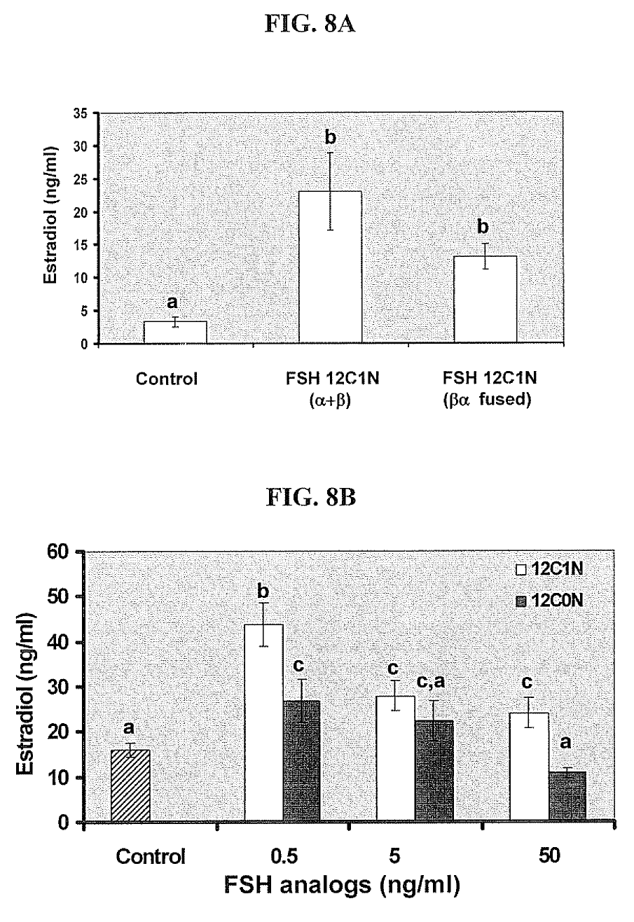

The gene encoding the piscine FSH β-subunit can be modified so as to provide glycosylation mutants. Muteins of piscine FSH β-subunit are prepared by deleting or adding N-linked glycosylation sites represented by triad amino acid sequons (NX^{S}/_{T}; N₁ and N₂ in FIG. 1) herein positioned at amino acids 12-14 and 27-29 of the mature BFT FSHβ peptide shown in FIG. 4B. Site-directed mutagenesis is performed on a piscine FSH β-subunit cDNA to disrupt or reconstitute the aforementioned sequons. The recombinant protein produced by a system capable of expressing the cDNAs encoding these muteins shows biological activity in terms of inducing estradiol secretion from ovarian tissues of various fish (FIG. 8A & FIG. 8B), and can be used as an agonist/antagonist for piscine FSH activity for therapeutic and/or diagnostic purposes.

Before presenting examples reference is made to the following materials and methods employed in the performance of experiments described in the examples.

### Materials and Methods

### Preparation Methods

Methods to construct the proteins of the invention are well known in the art. As set forth below, one approach is to synthesize the FSH analogs of the invention recombinantly by expression of the nucleotide sequence encoding the desired protein. A nucleic acid including the nucleotide sequence encoding the piscine FSH beta protein may be prepared from native sequences, or synthesized de novo or using combinations of these methods. Techniques for site-directed mutagenesis, ligation of additional sequences, PCR, and construction of suitable expression systems are all well known in the art. Portions or the entire DNA encoding for the desired protein can be constructed synthetically using standard solid phase techniques, preferably including restriction sites for ease of ligation. Suitable control elements for transcription and translation of the included coding sequence may be provided to the DNA coding sequences. As is well known, expression systems compatible with a wide variety of hosts, including procaryotic hosts such as bacteria and eucaryotic hosts such as yeast, fungi such as Aspergillus and Neurospora, plant cells, insect cells, mammalian cells such as CHO cells, avian cells, and the like, are available.

The piscine FSH analogs of the invention are most efficiently produced using recombinant methods, but may also be constructed using synthetic peptide techniques or other organic synthesis techniques known in the art.

The present invention is further embodied by a diagnostic kit for analysis of a biological sample removed from a subject. The kit includes reagents suitable for conducting a quantitative analysis of a piscine FSH expression level in the biological samples In other words, the kit facilitates practice of the diagnostic method.

According to still further features in the described preferred embodiment, the diagnostic kit further includes packaging material and instructions for performance of the quantitative analysis on at least one type of biological sample. The instructions, in a most preferred embodiment, further include an explanation of at least one method for collection of the biological sample from the subject. Optionally, but preferably, the kit further includes reagents for generation of standards for comparison. Most preferably the standard for comparison is a calibration curve.

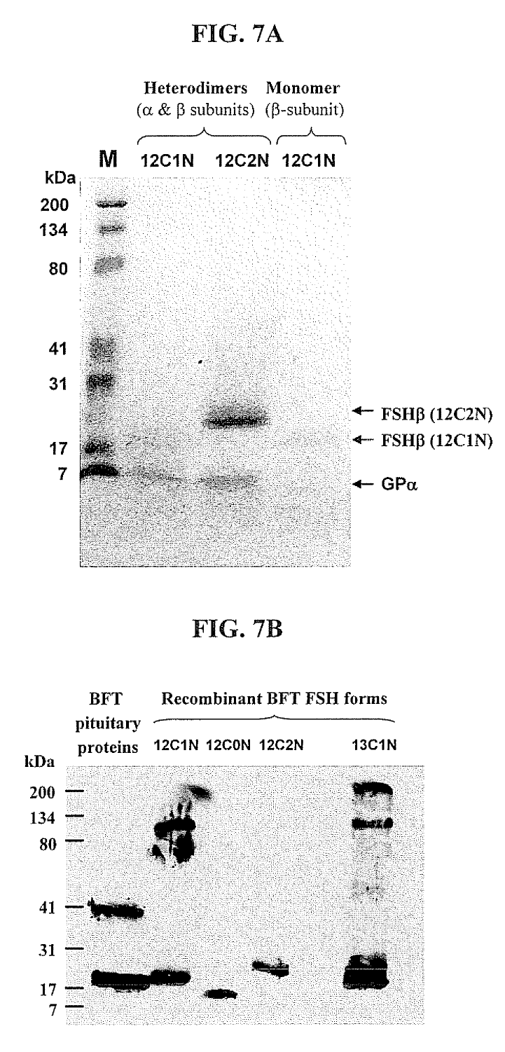

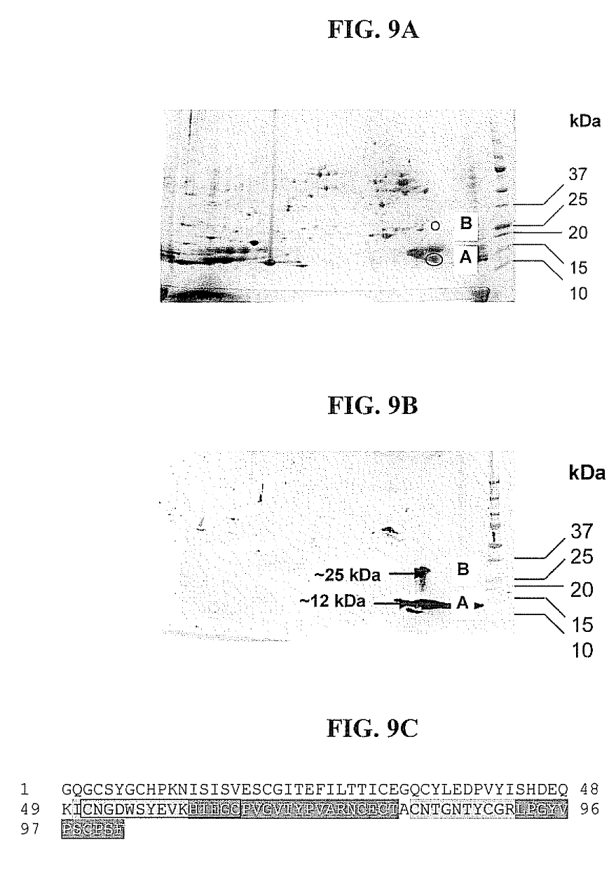

According to alternate preferred embodiments of the invention, the quantitative analysis of a piscine FSH expression level in a biological sample taken from the subject employs an antibody specific to at least a portion of the piscine FSH protein. Thus, the quantitative assay might be, for example, a western blot (see FIGS. 7B and 9B), ELISA (enzyme linked immunosorbent assay), immunohistochemistry or RIA (Radio immunoassay).

Practice of this method of treatment is preferably accomplished by administration of a pharmaceutical composition for expediting the onset of puberty in captive fish and/or to alleviate reproductive dysfunctions. The pharmaceutical composition further embodies the invention. The pharmaceutical composition includes as an active ingredient a physiologically effective amount of a piscine FSH agonist of the invention useful in treating reproductive disorders in fish, in admixture with at least one pharmaceutically acceptable carrier and/or excipient.

### Methods of Use

The recombinant FSH agonists/antagonists of the invention may be used as substitutes for piscine FSH in treatment of infertility, as aids in in vitro fertilization techniques, and other therapeutic methods associated with the wild type piscine hormone. The recombinant FSH agonists/antagonists of the invention may be employed as diagnostic tools to detect the presence or absence of antibodies with respect to the native FSH protein in biological samples. They are also useful as control reagents in assay kits for assessing the levels of FSH in various samples. Methods for measuring levels of the hormone itself or of antibodies effective against it are standard immunoassay protocols commonly known in the art. Various competitive and direct assay methods may be used involving a variety of labeling techniques including radio-isotope labeling, fluorescent labeling, enzyme labeling, and other known techniques.

The recombinant piscine FSH agonists/antagonists of the invention may also be used to detect and purify receptors to which the wild type hormone binds. Thus, the recombinant FSH agonists of the invention may be coupled to labels, solid supports, and the like, depending on the desired application. The proteins of the invention may be coupled to carriers to enhance their immunogenicity in the preparation of antibodies specifically immunoreactive with these new modified forms. When coupled, these proteins can then be used as affinity reagents for the separation of desired components with which specific reaction is exhibited. They may be used in affinity chromatographic preparation of receptors or antihormone antibodies. The resulting receptors are themselves useful in assessing hormone activity for candidate drugs in screening tests for therapeutic and reagent candidates.

Additionally, the antibodies uniquely reactive with the recombinant FSH agonists of the invention may be used as purification tools for isolation of subsequent preparations of these materials. They can also be used to monitor levels of the recombinant FSH agonists administered as drugs.

### Antibodies

The proteins of the invention may be used to generate antibodies specifically immunoreactive with these new proteins. These antibodies are useful in a variety of diagnostic and therapeutic applications. The antibodies are generally prepared using standard immunization protocols in mammals such as rabbits, mice, sheep or rats, and the antibodies are tittered as polyclonal antisera to assure adequate immunization. The polyclonal antisera call then be harvested as such for use in assays such as immunoassays. Antibody-secreting cells from the host may be immortalized using known techniques and screened for production of monoclonal antibodies immunospecific with the proteins of the invention.

### Utility and Administration

The hormones aid other pharmaceuticals of the present invention are formulated for administration using methods generally understood in the art. Typical formulations and modes of administration are described in Remington's Pharmaceutical Sciences, Mack Publishing Co., Easton, Pa., latest edition. These formulations are typically for systemic administration, such as by injection, but oral formulations or topical formulations may also be employed. The choice of formulation, mode of administration, and dosage level are dependent on the particular hormone or protein and can be optimized for the appropriate indication using generally recognized techniques. Optimization of dosage regimen and formulation is conducted as a routine matter and as generally known in the art.

For recombinant production, modified host cells using expression systems are used and cultured to produce the desired FSH glycoprotein. These terms are used herein as follows:

### Definitions

The term “piscine” as used herein means all kind of fishes, a group consisting of approximately 24,600 living species. Of these, 85 are jawless fishes (hagfishes and lampreys); 850 are cartilaginous (sharks, skates, rays, and chimaeras); and the vast majority are bony fishes (−23,000 species).

As used herein, piscine FSHβ and GPα subunits, as well as the heterodimeric form, generally have their conventional definitions and refer to the proteins having the amino acid sequences known in the art per se, or allelic variants thereof, purposely constructed muteins thereof having agonist/antagonist activity regardless of the glycosylation pattern exhibited. When only the beta chain is referred to, the term is specified as FSHβ. The term “FSH” refers to the heterodimer. The way the glycosylation pattern is affected by alteration of the glycosylation sites is evident from the context. Recombinant forms of the FSH glycoprotein with specified glycosylation patterns are noted.

“Native” forms of the FSHβ and GPα peptides are those that have the amino acid sequences isolated from the specific fish (e.g. bluefin tuna, BFT), and have these known sequences per se, or their allelic variants.

“Mutein” or “mutant” or “variant” forms of piscine FSH glycoprotein are those which have deliberate alterations, including insertion, deletions and/or truncations, in amino acid sequence of the native protein produced by, for example, site-specific mutagenesis or by other recombinant manipulations, or which are prepared synthetically. The mutants have altered N-linked glycosylation sites of the FSHβ subunit and varied number of cysteine residues. Preferably, the mutants the piscine FSH beta subunit have no, one, or at least 2 N-linked glycosylation sites and at least 12 cysteine residues. The mutants may comprise additional N-linked glycosylation sites and cysteine residues, such as 14 or 15 cysteine residues, so long as the alterations result in amino acid sequences wherein the biological activity of the subunit is retained. The GPα portion of the molecule is essentially constant, although minor variations are or may be present.

The terms “peptide” and “protein” are used interchangeably herein to refer to amino acid polymers in which one or more amino acid residues is an artificial chemical analogue of a corresponding naturally occurring amino acid, as well as to naturally occurring amino acid polymers.

Amino acids may be referred to herein by either their well known three letter symbols or by the one-letter symbols recommended by the IUPAC-IUB Biochemical Nomenclature Commission. Similarly, nucleotides are referred to by their commonly accepted single-letter codes. The term “amino acid residue” is intended to indicate any naturally or non-naturally occurring amino acid residue, in particular an amino acid residue contained in the group consisting of the 20 naturally occurring amino acids, i.e. alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or 1,), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues.

A “chimeric molecule” as used herein refers to a molecule obtained after conjugation of two or more different types of molecules (e.g., lipids, glycolipids, peptides, proteins, glycoproteins, carbohydrates, nucleic acids, natural products, synthetic compounds, organic molecule, inorganic molecule, etc.). In general, the nomenclature and the laboratory procedures in cell culture, molecular genetics, and nucleic acid chemistry used herein and described below are those well known and commonly used in the art. Standard techniques such as described in Sambrook et al., MOLECULAR CLONING: A LABORATORY MANUAL (Cold Spring Harbor Laboratory, Cold Spring Harbor, N.Y., 2nd ed. 1989) and CURRENT PROTOCOLS IN MOLECULAR BIOLOGY Vols. 1-3 (Virginia Benson Chanda ed. John Wiley & Sons, 1994-1998; and BIOCHEMISTRY WITH CLINICAL, CORRELATIONS (T. Devlin ed., 3d ed. 1992), each of which is incorporated herein by reference in its entirety for all purposes, are used for recombinant nucleic acid methods, nucleic acid synthesis, cell culture, and transgene incorporation, e.g., electroporation, injection, ingestion, and lipofection. Electroporation technique uses a pulse of high electrical current to introduce molecules of interest into cells, tissues, or organisms. Lipofection employs lipid-like cationic molecules that interact strongly with cell membranes, destabilizing them locally, thereby allowing DNA and RNA entry into cells. Generally, oligonucleotide synthesis and purification steps are performed according to the specifications provided. The techniques and procedures are generally performed according to conventional methods in the art and in accordance with various references specified herein.

A “physiological activity” in reference to an organism is defined herein as any normal processes, functions, or activities of a living organism. By “biological activity” is meant activity that is either agnostic or antagonistic to that of the native hormones.

A “therapeutic activity” is defined herein as any activity of e.g., an agent, gene, nucleic acid segment, pharmaceutical, therapeutic, substance, compound, or composition, which decreases or eliminates pathological signs or symptoms when administered to a subject exhibiting the pathology. The term “therapeutically useful” in reference to an agent means that the agent is useful in diminishing, decreasing, treating, or eliminating pathological signs or symptoms of a pathology or disease.

An “expression system” or “expression vector” refers to a nucleic acid molecule containing a nucleotide sequence that is expressed in a host cell. Typically, the expression vector is a DNA molecule containing a gene, and expression of the gene is under the control of regulatory elements that may, optionally, include one or more constitutive or inducible promoters, tissue-specific regulatory elements, and enhancers. Such a gene or nucleic acid sequence is said to be “operably linked to” the regulatory elements. The accompanying control DNA sequences necessary to effect the expression of the coding sequence typically include a promoter, termination regulating sequences, and, in some cases, an operator or other mechanism to regulate expression. The control sequences are those which are designed to be functional in a particular target recombinant host cell and therefore the host cell must be chosen so as to be compatible with the control sequences in the constructed expression system. The “expression vector” includes, but is not limited to plasmids, phage vectors, phagemids, cosmids, viral vectors (e.g. adenovirus or lentivirus vectors), and other vectors which are known or will become known to those familiar with recombinant nucleic acid technology. The scope of the invention further includes a cell transfected with such an expression vector. The gene expression vector is capable of delivery/transfer of heterologous nucleic acid into a host cell. The expression vector may include elements to control targeting, expression and transcription of the nucleic acid in a cell selective manner as is known in the art.

Expression vectors can be introduced into cells or tissues by any one of a variety of known methods within the art. Such methods can be found generally described in Sambrook et al., Molecular Cloning: A Laboratory Manual, Cold Springs Harbor Laboratory, New York 1989, 1992), in Ausubel et al., Current Protocols in Molecular Biology, John Wiley and Sons, Baltimore, Md. 1989), Chang et al., Somatic Gene Therapy, CRC Press, Ann Arbor, Mich. 1995). Vega et al., Gene Targeting, CRC Press, Ann Arbor Mich. (995), Vectors: A Survey of Molecular Cloning Vectors and Their Uses, Butterworths, Boston Mass. 1988) and Gilboa et al. (Biotechniques 4 (6): 504-512, 1986) and include, for example, stable or transient transfection, lipofection, electroporation and infection with recombinant viral vectors.

A “modified” recombinant host cell, i.e., a cell “modified to contain” the recombinant expression systems of the inventions refers to a host cell which has been altered to contain this expression system by any convenient manner of introducing it, including transfection, viral infection, and so forth. “Modified cells” refers to cells containing this expression system whether the system is integrated into the chromosome or is extrachromosomal. The “modified cells” may be either stable with respect to inclusion of the expression system or the encoding sequence may be transiently expressed. Recombinant host cells “modified” with lie expression system of the invention refers to cells which include this expression system as a result of their manipulation to include it, when they natively do not ordinarily do so, regardless of the manner this is accomplished.

A “transfected” recombinant host cell, item a cell “transfected” with the recombinant expression systems of the invention, refers to a host cell which has been altered to contain this expression system by any convenient manner of introducing it, including transfection, viral infection, and so forth. “Transfected” refers to cells containing this expression system whether the system is integrated into the chromosome or is extrachromosomal. The “transfected” cells may either be stable with respect to inclusion of the expression system or not. Thus, “transfected” recombinant host cells with the expression system of the invention refer to cells including this expression system as a result of being manipulated to include it, when they natively do not, regardless of the manner of effecting this incorporation. “Transformation” and “transfection” are used interchangeably to refer to the process of introducing DNA into a cell.

As used herein “cell”, “host cell”, “cell culture” and “cell line” are used interchangeably herein and all such terms should be understood to include progeny resulting from growth or culturing of a cell. Where the distinction between them is important, it will be clear from the context. Where any can be meant, all are intended to be included.

The term “polymerase chain reaction” or “PCR” refers to the well-known method for amplification of a desired nucleotide sequence in vitro using a thermostable DNA polymerase.

The term “nucleotide sequence” is intended to indicate a consecutive stretch of two or more nucleotide molecules The nucleotide sequence may be of genomic, cDNA, RNA, semisynthetic or synthetic origin, or any combination thereof.

A “cloning vector” is a nucleic acid molecule, typically a DNA molecule, having the ability to replicate autonomously in a host cell. The cloning vector can be, for example, a plasmid, cosmid, or bacteriophage, and may be linear or circular. Cloning vectors typically contain one or more restriction endonuclease recognition sites at which foreign nucleic acid sequences can be inserted in a determinable fashion without loss of an essential biological function of the vector, as well as a marker sequence that is appropriate for use in the identification and selection of cells transformed with the cloning vector. Marker genes typically include nucleic acid sequences that encode polypeptides that may confer a phenotypic characteristic to the transformed cell, such as antibiotic resistance, test compound metabolism, etc.

The term “mutagen” is understood as meaning any mutagenic or potentially mutagenic agent or event, including a mutagenic chemical compound, such as a toxicant, or exposure to radiation, including but not limited to alpha, beta, or gamma emissions from a radioisotope, electromagnetic radiation of any frequency, such as x-ray, ultraviolet, or infrared radiation, exposure to an electromagnetic field (EMF), and the like.

The protein produced may be recovered from the lysate of the cells if produced intracellularly, or from the medium if secreted. Techniques for recovering recombinant proteins from cell cultures are well known in the art. These proteins may be purified using known techniques such as chromatography, gel electrophoresis, selective precipitation, etc.

The term “glycosylation site” is used to refer to an N-linked glycosylation that requires a tripeptidyl sequence of the formula Asp-X-Ser or Asp-X-Thr, wherein X is any amino acid other than proline (Pro), which prevents glycosylation. An N-linked glycosylation site may be a tripeptidyl sequence of the formula Asn-X-Ser or Asn-X-Thr, wherein Asn is the acceptor and X is any of the twenty genetically encoded amino acids except Pro, which is known to prevent glycosylation. The removal of a glycosylation site is preferably achieved by amino acid substitution for at least one of the two critical residues (Asp or Ser/Thr) of the glycosylation signal. Alternatively, the term “glycosylation site” may refer to an O-linked glycosylation site which is not present normally in the FSHβ-subunit. The O-linked glycosylation structure (N-acetylgalactosamine residue is linked to the hydroxyl group of either a serine or threonine residue of a polypeptide) in its carboxyl terminal extension.

The term “pharmaceutical composition” refers to a preparation of one or more of the active ingredients described herein with other chemical components such as physiologically suitable carriers and excipients. The purpose of a pharmaceutical composition is to facilitate administration of a compound to an organism. Herein the term “active ingredient” refers to the peptide, protein, nucleic acids and/or antibodies accountable for the biological effect.

The phrases “physiologically acceptable carrier” and “pharmaceutically acceptable carrier” which may be interchangeably used to refer to a carrier or a diluent that does not cause significant irritation to an organism and does not abrogate the biological activity and properties of the administered compound. An adjuvant is included under these phrases.

The term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

All or a portion of the hormones of the invention may be synthesized directly using peptide synthesis techniques known in the art, and synthesized proteins may be ligated chemically or enzymatically.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples, which are intended to illustrate but not to limit the invention.

### EXAMPLE 1

### Piscine FSHβ Analog Design

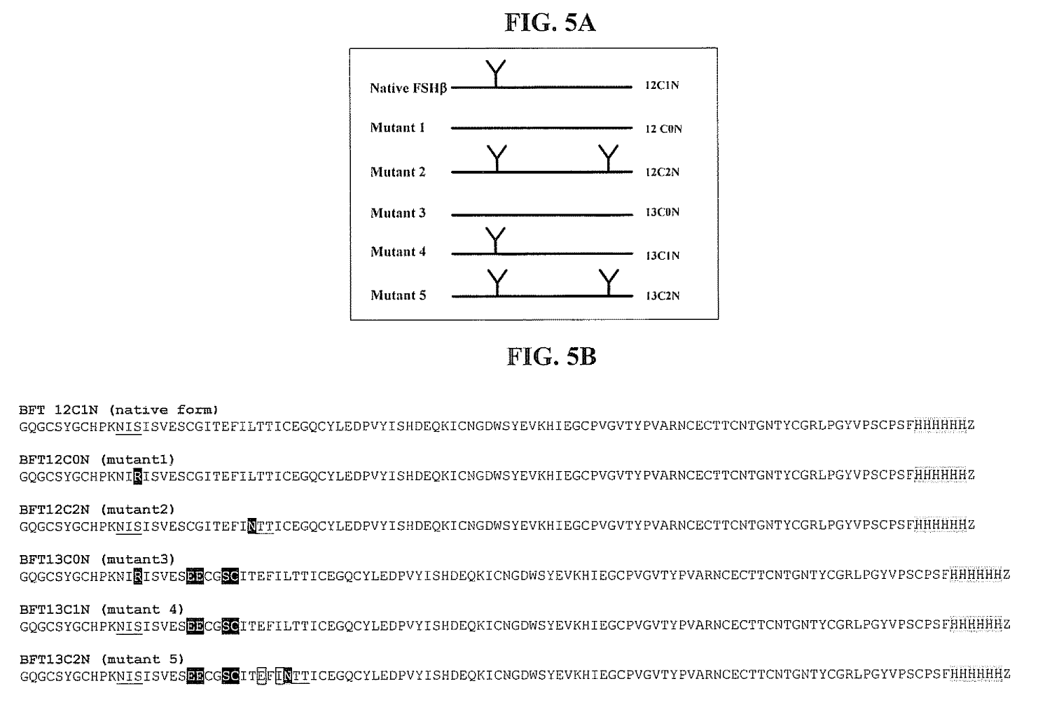

A series of FSHβ analogs with characteristic intramolecular disulfide bonds and glycosylation patterns was established, as shown in FIGS. 5A and 5B. The specific modifications are obtained by alteration of glycosylation sites normally present in the subunit through site directed mutagenesis at the appropriate amino acid residues. These include three forms of FSHβ consisting of the 12 cysteine backbone typifying teleosts, with varying number of N-linked glycosylation sites (e.g. 12C0N, 12C1N, 12C2N), as well as three forms of FSHβ consisting of the 13 cysteine backbone typifying cyprinid species with varying number of N-linked glycosylation sites (e.g. 13C0N, 13C1N, 13C2N). The cDNAs encompassing the respective mutations, and optimized codon usages for the expression in the heterologous host cells (Pichia pastoris; a methylotrophic yeast), were synthetically synthesized by GENEART (HY Laboratories LTD) according to amino acid sequence deduced from bluefin tuna (BFT) FSHβ cDNA (FIG. 4B).

The native FSHβ form includes 12 cysteine residues and one N-linked glycosylation site (12C1N). Site-directed mutagenesis was conducted on a teleost FSH beta subunit gene resulting in five mutant prototypes (FIG. 5):
Mutant 1, 12C0N: includes a single replacement of serine (S) with arginine (R) disrupting the only available putative N-linked glycosylation sequon (N₁; FIG. 1). Thus, mutant 1 includes 12 cysteine residues and totally lacks N-glycosylation sites.
Mutant 2, 12C2N: includes a single replacement of leucine (L) with asparagine (N) reconstituting the N₂ putative glycosylation sequon (FIG. 1). Thus, mutant 2 includes 12 cysteine residues and two N-linked glycosylation sites.
Mutant 3, 13C0N: includes a replacement of (S) with (R) (as in mutant 1) and two insertions of di-amino acids: (i) two tandem glutamic acids [EE], (ii) tandem serine and cysteine [SC]. Both insertions mimic the corresponding motif in catfish and carp FSHβ, which posses 13 cysteine residues (FIG. 1). Thus, mutant 3 includes 13 cysteine residues and lacks N-glycosylation sites.
Mutant 4, 13C1N: includes two insertions of di-amino acids EE and SC (as in mutant 3). Thus, mutant 4 includes 13 cysteine residues and one N-linked glycosylation site.
Mutant 5, 13C2N: includes a replacement of L with N (as in mutant 2), two insertions of di-amino acids FE and SC (as in mutant 3), and deletion of two amino acids: [E] and isoleucine [I], which mimics the corresponding motif in catfish. Thus, mutant 5 includes 13 cysteine residues and two N-linked glycosylation sites.

The FSH alpha subunit dimerizes with the recombinant FSH beta subunit to form the desired teleost FSH heterodimer. Such mutated sequences when ligated into expression systems and transfected into appropriate host cells result in production of proteins which, when combined with the appropriate alpha subunit have agonist activity for the relevant hormone.

### EXAMPLE2

### Construction of Expression Vectors for Piscine FSH and its Mutants

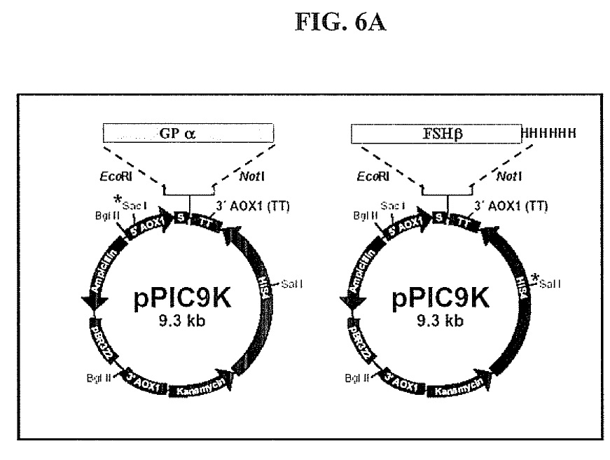

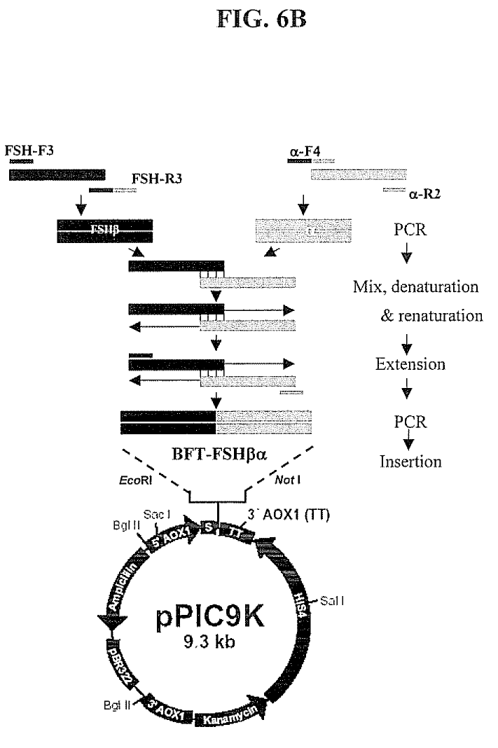

An expression system for piscine FSH beta-encoding DNA that provides FSH β-subunit that readily dimerizes to form bioactive piscine FSH hormone is shown in FIG. 6A.

The specific cDNA encoding for BFT FSH β-subunit was isolated using the SMART RACE cDNA amplification kit (Clontech), total RNA extracted from BET pituitary using TRIzole® (Gibco-BRL, Gaithersburg, USA) reagent, and gene specific primers (hereinafter “GSP”, shown in Table 3 below) For initial cloning of the 3′-end of BFT FSHβ cDNA two consecutive PCR reactions were performed with degenerate GSP (e.g. FSH-F1 and FSH-F2) that were designed according to amino acid sequences displaying high conservation among perciform species (i.e. Thunnus obesus-Okada et al., (1994) Int. J. Pept. Protein Res. 43: 69-80; bonito—Koide et al., (1993) Int. J. Pept Protein Res. 41: 52-65; striped bass—Hassin et al., (1995) Biol. Reprod. 58: 1233-1240; seabream—Elizur et al., (1996) Gen. Comp. Endocrinol. 102: 39-46). The isolated 3′-end amplicon (246 bp) was used to design a gene specific anti-sense primer (FSH-R1), with which the related 5′-end (456 bp) was cloned as well Superimposition of the 5′ and 3′ ends indicated that it comprises the full-length BFT FSHβ cDNA (562 bp) including: 5′ untranslated region (hereinafter “UTR”—139 bp), putative signal peptide (45 bp; 15 aa), mature peptide (309 bp; 103 aa) and 3′UTR (69 bp), as shown in FIG. 4B. Using the same approach, the full length cDNA sequence encoding for GPα subunit was isolated too (as shown in FIG. 4A).

The cDNAs encoding for the mature BFT FSHβ (309 bp) and GPα-subunit (282 bp) were PCR amplified using the respective set of primers FSH-F3/FSH-R2 and α-F3/α-R1 (shown in Table 3). To tag the recombinant protein the BFT-FSHβ anti-sense primer, FSH-R2 (Table 3) introduced a sequence codifying 6 histidine residues (6×His) flanked by a stop codon. Each PCR amplicon was independently introduced into the P pastoris expression vector, pPIC9K (Invitrogene), as an EcoRI/NotI insertion, 5′ flanked by the sequence coding for the yeast mating factor-α secretion signal [S]. FIG. 6A illustrates construction of expression vectors for the production of teleostean (e.g. bluefin tuna; BFT) FSHβ subunit and GPα-subunit chimeras for co-expression in P. pastoris.

Alternatively, a PCR product encompassing translational fusion of mature BFT FSHβ and GPα subunits (FIG. 4C) was subcloned into pPIC9K expression vector, as an EcoRI/NotI insertion (FIG. 6B). To facilitate homologous regions between the GPα and BFT FSHβ amplicons, the FSH-R3 primer (Table 3) introduced an extension of 15 bp, coding for the first 5 amino acids of GPα-subunit, whereas the α-F4 primer introduced a reciprocal extension, coding for the last 5 amino acids of the BFT-FSHβ. A mixture of both amplicon populations was denatured and then re-natured, allowing the association of homologous regions and the creation of a BFT-FSHβα (5′→3′) fusion (See FIG. 6B). In the latter case, the fused recombinant protein was tagged with 6×His at the C-terminus of GPα using α-R2 primer (Table 3).

TABLE 3
Gene Specific Primers Used to Clone the cDNA Sequences
Encoding for BFT FSHβ and GPα-Subunits

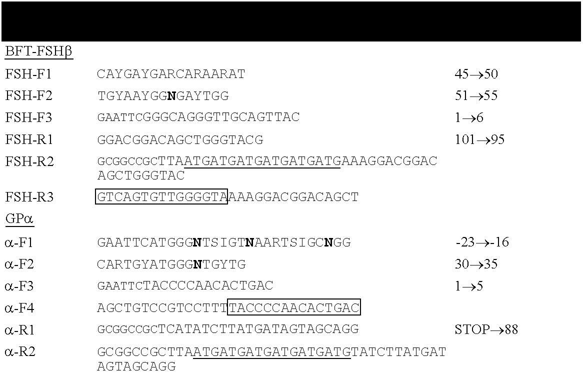

The identifications F and R denote primer direction: Forward (5→3′) and Reverse (3′→5′), respectively.
Underlined letters represent the additional 6 histidine codons.
Bold letters within primer sequences represent the following degeneracy: I-inosine; K-G or T; N- any of the four nucleotides (A/T/C/G); R- A or G; S- C or G; Y- C or T.
Small uppercase letters indicate the EcoRI and NotI restriction sites. Sequence codifying the first five amino acids is framed.

To facilitate integration of target genes to the yeast genome, the constructed plasmids were linearized with SacI or SallI (the respective recognition sites are marked with asterisks) before transforming the yeast cells.

In a manner similar to that set forth in Example 2, expression vectors for the production of mutants 1-5 of FSHβ (FIGS. 5A) set forth in Example 1, as a single subunit (FIG. 6A) or as a subunit translationally fused to the GPα-subunit (FIG. 6B), were prepared and used to transfect P. pastoris cells. The heterodimer FSH resulting from expression of these sequences had the biological activity of native FSH, The resulting hormones show activities similar to those of the wild-type form, when assayed as set forth in Example 5 below.

The foregoing constructions merely illustrate expression vectors or systems that may be constructed for the production of teleost FSH β-subunit or its mutants, and/or the GPα-subunit alone or as part of the corresponding heterodimeric FSH hormone. Alternate control sequences, including, for example, different promoters, can be ligated to the coding sequence of teleost FSH beta-subunit to effect expression in other eucaryotic cells that will provide suitable glycosylation.

### EXAMPLE 3

### Recombinant Protein Production

The constructed plasmids (5 μg), encompassing the BFT-FSHβ and GPα subunit, (FIG. 6A), were linearized with SalI and SacI, respectively, and were used to co-transform the host strain GS115 (auxotrophic for histidine; Invitrogen) by electroporation. The procedure was carried out by the MicroPulser Electroporation System (Bio-Rad) using the pulse parameters of 2 kV and 2.9 msec, as established by transformation efficiency tests. Following selection on histidine-deficient agar plates, geneticin hyper-resistance transformants were picked for further expression analysis. Similarly, the constructed plasmids encompassing the single chain BFT-FSHβα subunits (FIG. 6B) were linearized with SalI and used to transform the aforementioned yeast host cells.

Following methanol induction, P. pastoris transformants, resistant to higher levels (4 mg/ml) of geneticin, were screened using specific antibody (see below) for recombinant BFT-FSH expression,. Each selected colony was grown on buffered BMGY medium (1% yeast extract; 2% peptone; 100 mM potassium phosphate, pH 6.0; 1.34% yeast nitrogen base; 4×10-5% biotin; 1% glycerol) in a shaking incubator (250 rpm) at 28° C., for 2 days, The cells were harvested, re-suspended in buffered BMMY medium (same as BMGY but containing 1% methanol instead of 1% glycerol) to induce the AOX1 promoter and groWn for 3-4 days.

P. pastoris transformiants exhibiting higher expression level were picked for large-scale production of recombinant BFT-FSH. The culture superuatant was collected by centrifugation (1500 g, 10 min), and applied onto HiTrap chelating HP column (Amersham) for His-tagged protein purification.

### EXAMPLE 4

### Antibody Preparation Against Synthetic Peptide of BFT FSHβ

Unique peptide sequence corresponding to amino acid residues 50-65 of BFT FSHβ cDNA sequence (FIG. 4B), was synthetically synthesized (BioSight, Karmiel, Israel). The synthetic peptide was used as an antigen for the production of BFT-FSHβ specific antibodies in rabbits (Harlan Biotech Israel). The obtained antisera showed high specificity in Western blot analysis of BFT pituitary proteins (FIG. 9B). Total protein extracts were recovered from BFT pituitary and run on 2D-PAGE, using immobilized pH gradients for the first dimension (FIG. 9A). A Western blot analysis was preformed first with anti-FSHβ (FIG. 9B). The anti-FSH recognized two protein bands: a predominant band of about 12 kDa and a lesser band of about 25 kDa. The estimated molecular masses of the proteins recognized by the anti-FSHβ correlates well with the definite molecular masses of BFT FSHβ (12.95 kDa).

Considering the fact that molecular mass of the GPα-subunit is about 10 to 11 kDa, it seems that the additional recognized proteins of about 25 kDa represent traces of the FSH heterodimer. To further assess the anti-FSHβ specificity, the two highlighted spots (FIG. 9A; spots A and B), were robotically cut out from an equivalent 2D-PAGE stained with Commassie blue. The proteins were in-gel trypsinized and the resulting peptides were extracted, resolved by reversed phase capillary chromatography, and analyzed on-line by electrospray tandem mass spectrometry (Mass Spec). Using the NCBI protein database, both protein fractions were identified as tuna FSHβ. FIG. 9C demonstrates the sequences of the most frequent peptides found among spots A and B, as analyzed by the Mass Spec technique (Protein Analysis Center, Technion, Haifa, Israel). In addition, the aformentioned antibodies specifically recognize the recombinant mutein forms of BFT-FSH (FIG. 7B). BFT pituitary proteins (lane 1) and recombinant BFT FSH proteins (12C1N-lane 2; 12C0N-lane 3; 12C2N-lane 4; 13C1N-lane 5) were separated on SDS-PAGE (10-20% gradient) and analyzed by Western blotting using anti-BFT-FSHβ. The corresponding positions of the molecular mass (kDa) markers run simultaneously are indicated.

### EXAMPLE 5

### In vitro Bioactivity of BFT-FSH

The in vitro bioactivity of recombinant BFT-FSH was examined by its capacity to stimulate estradiol (E₂) secretion from ovarian follicles of various fish species (e.g. grey mullet and sea bass). For tis purpose, fish females undergoing final oocyte vitellogenesis were anesthetized in 0.07% clove oil and killed by decapitation Gonads were rapidly removed and placed in a cold incubation medium (75% Leibovitz L-15 medium with L-glutamine, and 0.1 g/ml gentamycine, pH 7.4). Then, uniformly sized pieces (average of 100±5 mg/piece) were preincubated using 24-well culture plate containing 1.5 ml of ice-cold incubation medium. Following three consecutive washes to eliminate endogenous steroids, the ovarian fragments were challenged (16 hours exposure) with fresh ice-cold medium containing graded doses of recombinant BFT-FSH or its mutant form, i.e., recombinant BET FSH 12C1N analogs consisting of independent or fused FSHβ- and GPα-subunits ([α+β] or [βα fused], respectively). When the experiment ended, media was collected, steroids were extracted twice with ethyl ether, and the E₂ levels were measured by specific ELISA elaborated for other fishes (Cuisset el al., 1994; Nash et al., 2000). Our results indicate that the recombinant BFT-FSH and its mutant fores significantly stimulate the release of E₂ from vitellogenic mullet (Mugil cephalus; FIG. 8A) and sea bass (Dicentrarchus labrax; FIG. 8B) ovaries as compared to the controls, pointing out the generic nature of the produced recombinant hormones. Moreover, the effect of the same dose (50 ng/ml) of recombinant BFT FSH 12C1N consisting of either independent FSHβ- and GPα-subunits (α+β) or a single chain BFT FSH 12C1N (βα) did not vary significantly (FIG. 8A), indicating functional resemblance of the two BFT FSH analogs Dose-response experiments performed with BFT FSH 12C1N (α+β) and its mutant form 12C0N (α+β), indicated greater bio-potency at the lowest concentration (0.5 ng/ml) for both analogs (FIG. 8B). The latter results coincide well with the relatively low levels of circulating FSH, so far, measured in salmonid fish (Prat et al., (1996) Biol. Reprod. 54: 1375-1382; Breton et al., (1998) Gen Comp Endocrinol 111: 38-50; Gomez et al., (1999) Gen. Comp. Endocrinol. 113: 413-428). Nevertheless, the 12C1N hormone was found to be significantly (P<0.05) more potent in inducing E2 release from sea bass ovarian fragments compared to 12C0N.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A mutein of piscine follicle stimulating hormone (FSH) β-subunit having an at least 12 cysteine residue backbone and having a modifed N-linked glycosylation pattern due to alteration of at least one N-linked glycosylation site of the native piscine FSH β-subunit nucleotide sequence, wherein the alteration is selected from the group consisting of a deletion of at least one N-linked glycosylation site and an addition of at least one N-linked glycosylation site.

2. The mutein of claim 1 having a 13 cysteine residue backbone.

3. A heterodimer comprising the mutein of claim 1 in combination with piscine glycoprotein α-subunit, wherein said heterodimer is an agonist or antagonist to the corresponding native piscine FSH gonadotropin hormone.

4. In a method which comprises utilizing piscine FSH to enhance fertility in captive fish, the improvement which comprises substituting for said FSH the heterodimer of claim 3.

5. A recombinant nucleic acid which encodes the mutein of claim 1.

6. The recombinant nucleic acid of claim 5 which further is capable of expressing a second nucleotide sequence encoding the piscine glycoprotein α-subunit.

7. An expression system for production of a mutein of claim 1 which expression system comprises a nucleotide sequence encoding the mutein of claim 1 operably linked to control sequences for its expression operable in a recombinant host cell to effect expression in said host cell.

8. Recombinant host cells modified to contain the expression system of claim 7.

9. The host cells of claim 8 which further comprise an expression system for the production of piscine glycoprotein α-subunit.

10. An expression system for production of an agonist or antagonist of piscine FSH which expression system comprises a first nucleotide sequence encoding the mute in of claim 1 operably linked to control sequences capable of effecting the expression of said first nucleotide sequence in a host cell and a second nucleotide sequence encoding piscine glycoprotein α-subunit operably linked to control sequences capable of effecting the expression of said second nucleotide sequence in the host cell.

11. Recombinant host cells modified to contain the expression system of claim 10.

12. A method to produce a mutein of claim 1 which method comprises culturing the cells of claim 8 under conditions wherein said mutein is produced and recovering the mutein from the cell culture.

13. A method to produce a mutein of claim 1 which method comprises culturing the host cells of claim 9 under conditions wherein said mutein is produced and recovering the mutein from the cell culture.

14. A method to produce recombinant piscine FSH hormone, which method comprises culturing the host cells of claim 11 under conditions wherein the nucleotide sequences encoding the piscine FSH a and β-subunits are expressed and recovering the hormone from the host cell culture.

15. A therapeutic composition which regulates the FSH concentration in piscine comprising an effective amount of the piscine FSH mutein of claim 1 useful in expediting the onset of puberty and treating reproductive disorders in fish, in admixture with at least one physiologically acceptable carrier, diluent, excipient or adjuvant.

16. A diagnostic kit for assessing the levels of piscine FSH in a sample that comprises:
(a) antibodies immunospecific to the mutein of claim 1; and
(b) at least one control reagent comprising said mutein of claim 1 to which said antibodies are immnunospecific.

17. A method of using the diagnostic kit of claim 16 to assess the amount of FSH in a piscine subject comprising the steps of: contacting a sample from said subject with the antibodies as provided in the diagnostic kit under conditions to obtain an observable first result; contacting the control reagent as provided in the diagnostic kit with said antibodies under conditions to obtain an observable second result; and comparing the first result with the second result.

18. A mutein of piscine follicle stimulating hormone (FSH) β-subunit having an at least 12 cysteine residue backbone and having a modifed glycosylation pattern due to alteration of at least one glycosylation site of the native piscine FSH β-subunit nucleotide sequence, wherein the alteration is selected from the group consisting of a deletion of at least one N-linked glycosylation site, an addition of at least one N-linked glycosylation site, and an addition of at least one O-linked glycosylation site.

19. A heterodimer comprising the mutein of claim 18 in combination with piscine glycoprotein α-subunit, wherein said heterodimer is al agonist or antagonist to the corresponding native piscine FSH gonadotropin hormone.

20. In a method which comprises utilizing piscine FSH to enhance fertility in captive fish, the improvement which comprises substituting for said FSH the heterodimer of claim 18.
